(19) United States
(12) Patent Application Publication
(10) Pub. No.: US 2010/0087804 A1
(43) Pub. Date: Apr. 8, 2010

(54) SYSTEM AND METHOD FOR TREATING TISSUE

(76) Inventors: Ofer Fridman, Bikat Beit Hakerem (IL); Ishay Attar, Haifa (IL); Ziv Attar, Zihron Yakkov (IL)

Correspondence Address: ABELMAN, FRAYNE & SCHWAB, 666 THIRD AVENUE, 10TH FLOOR, NEW YORK, NY, 10017, US

(21) Appl. No.: 12/312,280

(22) PCT Filed: Nov. 1, 2007
(86) PCT No.: PCT/IL2007/001339

§ 371 Date: Dec. 2, 2009

Related U.S. Application Data
(60) Provisional application No. 60/863,818, filed on Nov. 1, 2006.

(51) Int. Cl. A61B 18/20; A61B 17/03

(52) U.S. Cl. 606/11; 606/214

(57) ABSTRACT

A method and system for tissue treatment, wherein the method includes transmitting energy onto tissue to be treated, and maintaining a required distance, during the tissue treatment, between a hand piece and the tissue. The method may further include applying a soldering agent for facilitating tissue soldering, used as adhesive to various types of protein in human tissue, wherein the transmitting energy activates the soldering agent applied on the tissue.

### FIELD OF THE INVENTION

The present invention relates generally to a system and method for treating tissue and, more particularly, to a system and method for soldering or cutting tissue.

### BACKGROUND OF THE INVENTION

The mechanisms of tissue closure typically used after surgeries, injury, also referred to hereinafter as wound closuring, are well known surgical techniques. The tissue is connected together with suturing materials, such as silk, synthetic thread, or metal staples, and then allowed to permanently bind together by a natural healing process. One of the drawbacks of joining tissue with suturing materials is the introduction of foreign materials into the tissue. The foreign materials may cause inflammation, infection and scarring. Additionally, bonding tissue together with suturing materials does not necessarily create a tight, smooth seal, and may result in a non-aesthetic appearance, which may be highly significant when dealing with skin tissue in a strategic location in the body, such as face. Moreover, tissue which is bonded with a suturing material is not necessarily fluid-resistant, therefore is exposed to infections.

Due to the drawbacks of conventional methods for wound closure described above, wound soldering techniques were developed, wherein a variety of laser systems have been introduced into the market for providing soldering of a wound in the tissue.

Different kinds of lasers are used nowadays for tissue soldering, including CO₂ (10.6 μm wavelength), Er:Yag (Erbium Yttrium Aluminum Garnet, 2.94 μm wavelength) in the mid infrared range (IR), Nd (Neodymium) YAG (1064 nm wavelength) in the near IR and Argon ion, having 2 main lines (488 nm and 514 nm wavelengths) in the blue and green regions of the visible spectrum. The energy of the CO₂ and the Er:YAG lasers can substantially be absorbed in water and in soft tissue, which is comprised of 70-90% water, as well. Er:YAG laser is used to evaporate both soft and hard tissues. Each one of the laser types mentioned above is generally understood to cause tissue heating that produces structural changes in tissue, thereby causing cross-linking of proteins, also referred to hereinafter as denaturation, and binding of the tissue. As currently practiced, the laser is directed onto the tissue for a specified period of time at a specified power until subjective and visible tissue changes occur. Such changes may include blanching, browning or shrinking. Some evidence suggests that the soldering effect may vary depending upon which type of laser is used and on what type of tissue it is used on. For example, heating blood vessels with an Nd:YAG laser produces a soldering effect as a result of collagen inter digitation. In this process, the collagen fibrils develop a change in periodicity but are still recognizable. Similarly, Argon laser soldering is thought to stem from a structural change in the soldered tissues.

The differences between the laser types manifest in the extent of the damage caused to the surrounding tissue areas. For example, Argon and Nd:YAG lasers penetrate deep through the tissue while heating it, while a CO₂ laser heats the tissue with only superficial penetration. Accordingly, in soft tissue, such as the bladder, applying CO₂ laser energy onto the wound's edges surface, penetrating to a depth of approximately 0.1 mm, dramatically reduces the damage to surrounding tissue area as compared to a treatment with Aragon laser energy, which penetrates up to 1 mm in depth or Nd:YAG lasers energy, which penetrates up to the extent of 5 mm in depth. Consequently, the mechanism of tissue soldering by a CO₂ laser is rather different as compared to the mechanism when using the other types of laser mentioned previously. The CO₂ laser energy heats the tissue's surface, damaging it to the extent that the tissue produces a coagulum that forms a seal between the desired joint's edges. Therefore, the localized effect of a CO₂ laser makes it eminently suitable for tissue soldering, as the soldering process is rapid and safer to the adjacent areas.

The use of a protein soldering agent has been successfully demonstrated, as documented in several studies: Poppas, D., Schlossberg, S., Richmond, I., et al: “LASER WELDING IN URETHRAL SURGERY: IMPROVED RESULTS WITH A PROTEIN SOLDER” The Journal of Urology, Vol. 139, February 1988, pages 415-417; and Ganesan, G., Poppas, D., Devine, C.: “URETHRAL RECONSTRUCTION USING THE CARBON DIOXIDE LASER: AN EXPERIMENTAL EVALUATION” The Journal of Urology, Vol. 142, October 1989, pages 1139-1141.

An advantageous system would solder a wound after defining the area to be treated, aligning the edges of the wound together to insure safe and successful tissue soldering. An advantageous system would create a high-strength, fluid-resistant bond, employing a protein soldering agent. An advantageous system would create a bond which provides a bacterial barrier for the wound and thereby reduces bacterial contamination.

### SUMMARY OF THE INVENTION

The present invention relates to a method and system for tissue treatment including transmitting energy onto tissue to be treated, and maintaining a required distance, during the tissue treatment, between a hand piece and the tissue. The method may further include applying a soldering agent for facilitating in tissue soldering, used as adhesive to various types of protein in a tissue, wherein the transmitting energy activates the soldering agent applied onto the tissue. The method according to a further embodiment of the present invention includes determining the energy flux of the energy at the tissue and adjusting the energy flux applied onto the tissue. The method according to a further embodiment of the present invention includes calculating the energy flux and calculating adjustments required so as to cause calculated energy flux to approach a pre-defined energy flux. The method according to a further embodiment of the present invention includes determining the flux of the soldering substance and adjusting the soldering substance flux applied onto the tissue. The method according to a further embodiment of the present invention includes calculating the soldering substance flux applied onto the soldered tissue and calculating adjustments required so as to cause calculated substance flux to approach a pre-defined substance flux. The method according to a further embodiment of the present invention includes the step of moving the hand piece along the tissue according to a required pace, during the treatment, while the maintaining a required distance. The method according to a further embodiment of the present invention includes regulating a treating beam spot size for the adjustments of the energy flux. The method according to a farther embodiment of the present invention includes pressing the edges of the tissue together for a soldering procedure or apart for a cutting procedure.

A system for treating a tissue including an energy source, arranged to transmit energy onto tissue to be treated, means for maintaining a required distance during the tissue treatment between a hand piece which includes an outlet for the energy source and the tissue. The system according to a further embodiment of the present invention includes a spreader for applying a soldering substance for facilitating tissue soldering, used as adhesive to various types of protein in tissue, wherein the energy source activates a soldering agent applied onto the tissue, wherein the hand piece further includes an outlet for the spreader. The system according to a further embodiment of the present invention includes means for determining the energy flux of the energy projected onto the tissue and means for adjusting the energy flux applied onto the tissue. The system according to a further embodiment of the present invention includes a computerized control unit which calculates the energy flux applied onto the soldered tissue and calculates the adjustments required so as to cause calculated energy flux to approach a pre-defined energy flux. The system according to a further embodiment of the present invention includes means for determining the soldering substance flux and means for adjusting the soldering substance flux applied onto the tissue.

The system according to a further embodiment of the present invention includes a computerized control unit which calculates the soldering substance flux applied onto the soldered tissue and calculates the adjustments required so as to cause calculated substance flux to approach a pre-defined substance flux. The system according to a further embodiment of the present invention wherein the means for adjusting includes regulating the spreader outlet size and/or regulating the soldering substance flow rate and/or controlling the pace of the hand piece movements and/or controlling the distance between the hand piece and the tissue.

The system according to a further embodiment of the present invention wherein the means for maintaining are movable means which moves according to a required pace during the tissue treatment. The system according to a further embodiment of the present invention wherein the moveable means are slanted in a way which enables them to press the edges of the tissue together for a soldering procedure or apart for a cutting procedure.

The system according to a further embodiment of the present invention wherein the required pace is controlled by a pace-motor such that the system will provide desired treatment pattern. The system according to a further embodiment of the present invention, wherein the desired pattern is a uniform soldering pattern or a none-uniform soldering pattern in a predetermined soldering distribution. The system according to a further embodiment of the present invention further includes an optical unit, coupled to the energy source for projecting a treating beam onto a surface of the tissue and regulating a treating beam spot size. The system, according to a further embodiment of the present invention further includes an apparatus for heating the soldering substance flow to a required temperature while it is exiting the hand piece towards the surface of the tissue. The apparatus may heat the soldering substance by heating a coil at the tip of the spreader so as to activate the soldering substance. The heating of the soldering substance can be accomplished by a flow of hot air.

### BRIEF DESCRIPTION OF THE DRAWINGS

The invention is herein described, by way of example only, with reference to the accompanying drawings, wherein:

FIGS. 1a and 1b illustrate side and top views, respectively, of a first configuration of a soldering system according to an embodiment of the present invention, employing sliders; and

FIGS. 2a and 2b illustrate side and top views, respectively, of a soldering system according to a second embodiment of the present invention, employing motorized wheels; and

FIGS. 3a and 3b illustrate side and top views, respectively, of a soldering system, according to a third embodiment of the present invention, employing motorized wheels and sliders; and

FIG. 4 is a schematic side view of a soldering system, according to a fourth embodiment of the present invention, employing an imaging sensor and/or a distance sensor for measuring the distance between the tissue and a reference point on the hand piece; and

FIG. 5 is a schematic side view of a soldering system, according to a fifth embodiment of the present invention, employing a wound dressing apparatus which provides long time strength; and

FIG. 6 is a schematic side view of a soldering system, according to a sixth embodiment of the present invention, employing a temperature-controlled soldering agent apparatus; and

FIG. 7 is a schematic side view of a temperature-controlled soldering agent apparatus, according to an embodiment of the present invention.

### DETAILED DESCRIPTION OF THE PREFERRED EMBODIMENTS

The present invention relates to a system and method for treating tissue with heat. The treatment can be soldering a tissue and may be applied in a variety of medical procedures, including hemostasis. The soldering of a tissue provides strong tissue joining while considerably reducing the damage to surrounding tissue areas. The treatment can also be cutting a tissue. The tissue may be of an external organ, such as the skin, or of an internal organ, such as a blood vessel. In a preferred embodiment of the present invention, the type of tissue being treating is skin. One aspect of the present invention relates to a system and method for wound soldering using a soldering substance applied on the wound's surface. Activating energy is applied to the tissue's surface, after it was covered by the soldering agent, such as an albumin solution or other conventional soldering agent, to facilitate tissue soldering. These soldering agents act as adhesive to various types of protein in the tissue to provide reliable closure of the wound's edges.

The mechanism of tissue soldering is complex, as there are many variables to be adjusted and modified in the soldering process. The success of connecting two edges of tissue depends on several factors. First, it is necessary to align the edges of the tissue closely together without tension. Second, it is necessary to accurately estimate the shape of the wound's edges, so that soldering energy can be equally distributed to all edges. Third, since soldering tissue will partly destroy segments in the tissue's edges, it is important to solder enough tissue in order to avoid reopening of the weld in the future. Fourth, it has been found that real-time adjustment of the laser's parameters, such as focusing and/or power, may maximize the soldering effect and minimize peripheral tissue destruction. This is described in Applicants' co-pending PCT patent application entitled “SYSTEM AND METHOD FOR TISSUE SOLDERING”, filed on even date.

The energy transmitted for heating and activation of the soldering agent, as well as for soldering the wound, may be transmitted by a laser transmitter or any other appropriate transmitter, such as microwave transmitter, IR transmitter, Near IR transmitter, UV transmitter, Ultrasound transmitter and the like. A laser transmitter is described below as the preferred energy source, however the invention is not limited to such a source.

In previously reported pre-clinical and clinical tests of tissue bonding accomplished by using temperature-controlled laser soldering systems, extreme care was taken to manually maintain the distance between the laser and tissue, thus minimizing fluctuations in soldering beam spot size. Nonetheless, bonding inconsistencies were observed. In a real-life surgical environment, the application difficulties are greatly multiplied and consistent bonding along the length of a joint is nearly impossible.

A number of novel, innovative configurations of a system for treating tissue are described below. While each embodiment is described principally as a soldering system, including providing hemostasis, it will be appreciated that each may be also be used for cladding, dissecting or cutting of the tissue. Together, in various combinations, they enable the medical use of laser soldering, cladding and cutting by surgical or other medical professionals.

The principles and operation of the system and methods for treating tissue, according to the present invention, may be better understood with reference to the drawings and the accompanying description.

Before explaining embodiments of the invention in detail, it is to be understood that the invention is not limited in its application to the details of design and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. Further, where considered appropriate, reference numerals may be repeated among the figures to indicate corresponding or analogous elements.

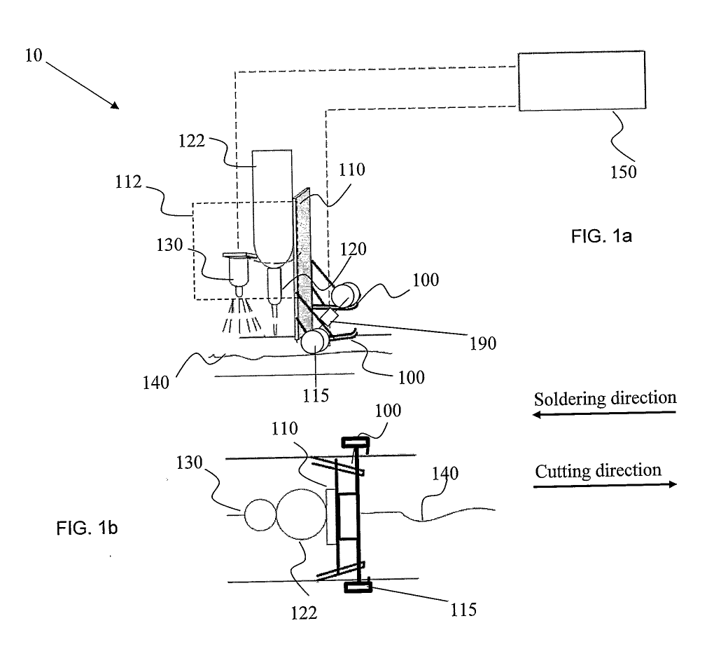

Reference is made to FIGS. 1a and 1b, illustrating a first configuration of a soldering system, according to one embodiment of the invention, employing sliders. This configuration is referred to, hereinafter, as the sliding soldering system (10). Sliding soldering system (10) includes a soldering energy source (122) providing soldering energy to a hand piece (112) for projection as a soldering beam spot onto a tissue to be soldered. At least two sliders (100), each one preferably having rotating means (115) are connected to hand piece (112) of soldering system, as by means of a holding unit (110). Typically, the hand piece (112) includes an energy source outlet (120), a soldering agent spreader (130) and an optional optical unit coupled to the soldering energy source, for regulating the soldering beam spot size. The sliders (100) are employed for aligning the edges of a wound (140) together in a way that enables tissue fixation prior to and during the soldering process, to ensure an accurate sealing of the wound. More specifically, the sliding soldering system (10) proceeds according to the following steps: The soldering agent is spread by a spreader, such as a sprayer or a brush, onto a desired tissue's surface, typically an open wound's surface. Soldering activating energy is applied to the desired tissue's surface, covered by the soldering agent, so as to activate the adhesion substance in the soldering agent. The sliders (100) assist in closing the edges of the treated surface, typically the edges of a wound. The steps mentioned above form a leak-tight seal of the wound, which is absorbed in the tissue, having two significant advantages, reducing bacterial contamination and aesthetically covering the wound. In another embodiment of the first configuration, if the wound is very narrow, approximately between 0.1 mm-0.2 mm, the sliders (100) may just move along both edges of the wound, without pressing the edges together.

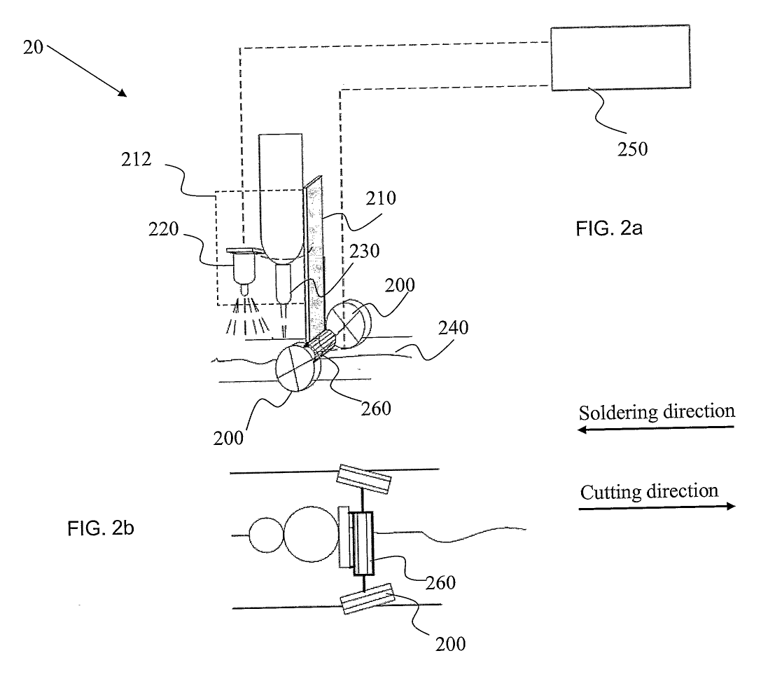

The soldering hand piece is preferably connected to the sliders (100) by a holding unit (110) which assists in maintaining a constant soldering beam angle. According to a preferred embodiment of the invention, the soldering energy source is a laser, most preferably a CO₂ laser for soft tissue. Alternatively, any other suitable heat source for providing focusable heat energy for treating a tissue may be utilized. The sliders (100) also assist in maintaining a required spreader-tissue distance and a required distance between the energy source outlet and the tissue's surface. Preferably, the sliders (100) are slanted in a way that, when the system (10) moves in a soldering direction, the slides (100) urge the tissue's edges together to facilitate closing the edges of the wound. The pace of system (10) may be controlled by a pace-motor (190), which receives signals from a computing unit (150) and activate the rotating means (115) accordingly. A specific description of the pace motor (190) is described with reference to FIGS. 2a and 2b.

Alternatively, the system illustrated in this embodiment can be used for cutting or dissecting tissue. In this case, the energy source acts so as to cut the tissue, rather than solder it. In operation, system (10) moves in a cutting direction, opposite to the soldering direction, so that slides (100) will separate the tissue's edges, urging them apart. The cutting is preferably accomplished by energy pulses in a frequency between 500 HZ to 1500 HZ and in an amplitude between 20 Watt to 50 Watt.

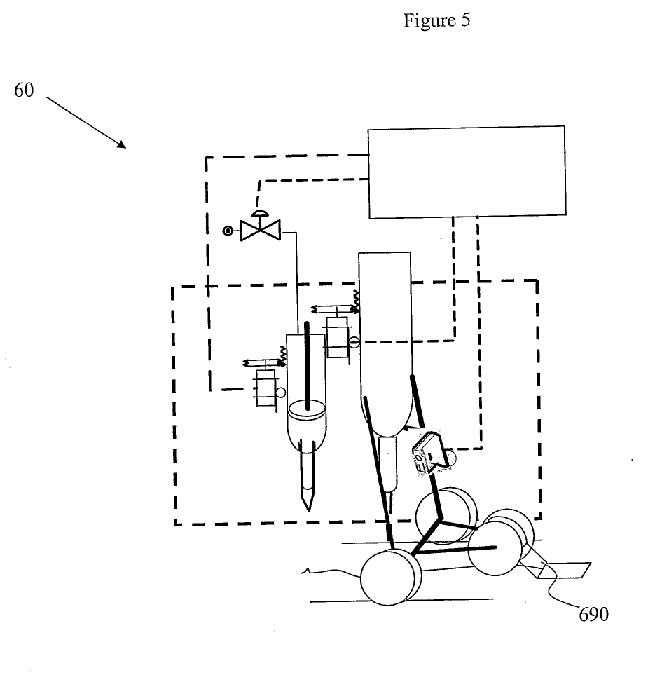

In one preferred embodiment of the sliding soldering system (10), a soldering protein, such as albumin or other type of soldering agent, is used to enhance bonding strength and minimize collateral damage to tissue. The soldering agent may be applied manually to the tissue in water-based solution form prior to the soldering process or in a preferred method wherein the soldering agent is applied automatically employing a control unit (150) for regulating the size of the soldering agent spreader's outlet and/or the flow rate of the soldering agent spreader and/or the distance between the soldering agent spreader's outlet and the tissue surface. For example, when increasing the distance between the spreader's outlet and the tissue surface, the sprayed distribution area grows, resulting in a decrease of the amount of soldering agent applied to the tissue per unit area, referred to hereinafter as the agent flux. For another example, when increasing the size of the soldering agent spreader's outlet and/or the flow rate of the soldering agent spreader, the agent flux grows. The control unit receives data of the sliding system's pace and the distance between a reference point on the soldering agent spreader and the surface of a tissue, also referred to hereinafter as the spreader-tissue distance. These two parameters may be constant or variable, depending on the requirements of the soldering procedure. The computing unit (150) constantly calculates the required changes in agent flux needed for obtaining desired agent flux and sends signals to an appropriate controlling mechanism, described with reference to FIG. 5.

The sliders (100) are preferably slanted in a direction which enables them to press the tissue inwards during the soldering process, to provide mechanical closure of the soldered tissue. When system (10) is used for cutting, the cutting direction is opposite to the soldering direction. The cutting procedure proceeds according to the following steps: the slanted sliders stretch uncut tissue and/or push the tissue's cut edges apart, and energy is applied to an uncut region of the tissue's surface so as to cut it.

Reference is made to FIGS. 2a and 2b, illustrating a second configuration of a soldering system, employing motorized wheels (200) instead of sliders, referred to hereinafter as the rolling soldering system (20). The wheels (200) fulfill the same purpose as the sliders mentioned with reference to FIG. 1: aligning the edges of a wound (240) together in a way that enables tissue fixation prior to and during the soldering or cutting process, to ensure accurate sealing of the wound or slicing of the tissue.

The system's hand piece (212) includes a spreader (220) and an outlet of an energy source (230) and is preferably connected by a holding unit (210), to a pace-motor (260), responsible for the pace of the rolling soldering system (20). The wheels (200) roll along the designated tissue's surface in a manner that urges the tissue edges in the desired direction. More specifically, during soldering, the rolling soldering system (20) proceeds according to the following steps. The soldering agent is sprayed onto a desired tissue surface by a spreader (220), typically an open wound's surface. A soldering energy is applied, through an energy outlet (230), onto the desired tissue surface, preferably already covered by the soldering agent, so as to heat the desired surface and/or cause activation of the soldering agent. The wheels (200) assist in closing the edges of the treated surface, typically the edges of a wound, providing top-coverage biological sealing. These steps can be carried out in any desired order. The wheels (200) are preferably slanted in a way which is similar to the sliders mentioned with reference to FIG. 1. The pace of system (20) may be controlled by a pace-motor (260), which receives signals from a computing unit (250) and translates them to motion. Specifically, the pace-motor (260) rotates the wheels (200) according to the translated signals. Preferably, the computing unit (250) automatically controls the pace of the rolling soldering system (20) according to the spreader-tissue distance. The computing unit (250) calculates the pace required such that the system will provide uniform soldering along the treated tissue. In another embodiment of the second configuration, the computing unit (250) calculates the pace required such that the system will provide non-uniform soldering, according to a predetermined distribution along the treated tissue.

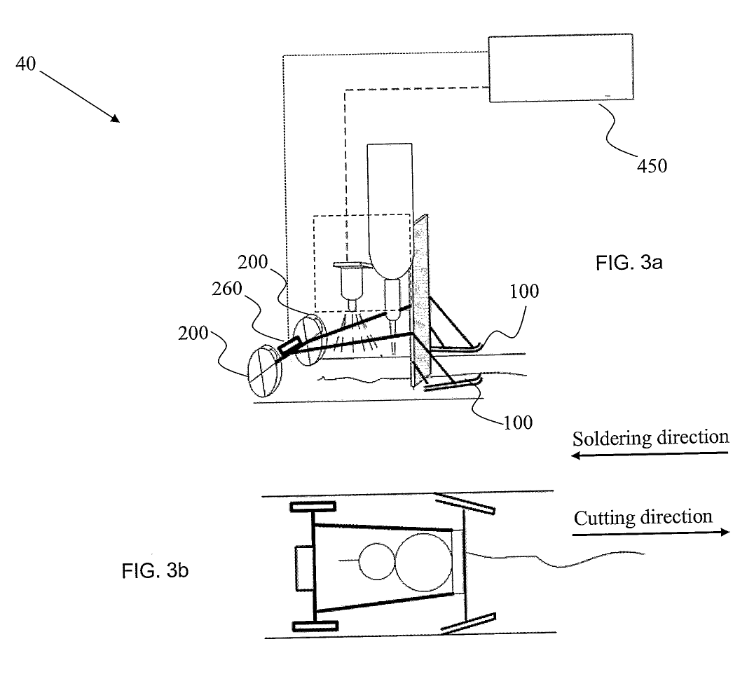

Reference is made to FIG. 3, illustrating a third configuration of a soldering system, employing wheels and sliders, referred to hereinafter as the sliding-rolling soldering system (40). Sliding-rolling soldering system (40) includes both a set of sliders (100) and a set of motorized wheels (200). This combination allows steady movement of the system, thereby providing highly accurate tissue treatment. Operation of this embodiment is substantially similar to the above descriptions with reference to FIG. 1a and FIG. 2a. The front wheels (200) and the back sliders (100) greatly assist in maintaining a required spreader-tissue distance and a required distance between the energy source outlet and the tissue's surface. The computing unit (450) sends signals to a pace-motor (260), which rotates the wheels (200) accordingly. The wheels of the sliding-rolling soldering system (40) are preferably not slanted, while the sliders (100) are preferably slanted in a similar manner as described with reference to FIG. 1. The sliders (100) press the wound's edges together during a soldering procedure or alternatively, push the tissue's surface apart in a cutting procedure.

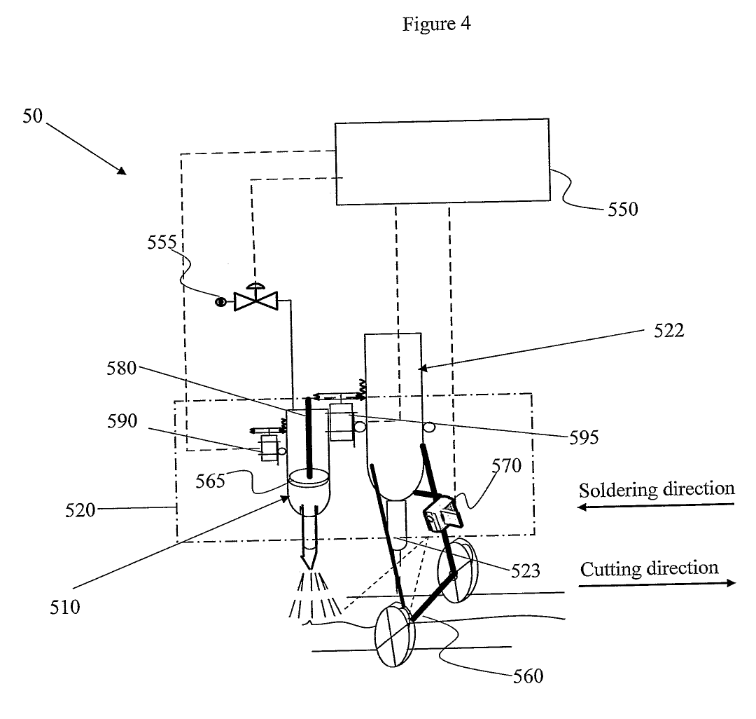

Reference is made to FIG. 4, illustrating a fourth configuration of a soldering system, employing an imaging sensor (570) and/or a distance sensor, called hereinafter the distance-controlled soldering system (50). Distance-controlled soldering system (50) includes a power source (522) transmitting energy through a hand piece (520) mounted on at least two wheels (560). System (50) may include a soldering agent spreader (510) in a hand piece (520). This embodiment further includes an additional apparatus (570) for determining the distance between the soldering agent spreader outlet and the tissue and the distance between the energy source outlet and the tissue, both distances are preferably the same distance. The additional apparatus (570) is an imaging sensor and/or a distance sensor coupled to a computing unit (550). In operation, imaging sensor (570) images an energy beam spot received on the tissue, exiting the hand piece (520), preferably a laser beam spot, and transfers the images to computing unit (550). Computing unit (550) processes the images and determines the spreader-tissue distance. Alternatively, or in addition, a distance sensor can be utilized, which simply measures the spreader-tissue distance and sends this data to computing unit (550). The distance sensor and the imaging sensor may be employed in the same system to provide highly accurate results.

The system's pace is measured by a tachometer or by a step motor controller also referred to as a pace-motor (560), an instrument that measures the rotation speed of a shaft or disk, as in a pace-motor. The spreader-tissue distance is determined by the apparatus for determining the spreader-tissue distance (570). The computing unit (550) constantly calculates the required changes in agent flux needed for obtaining desired agent flux. The required agent flux's changes are automatically transferred as signals to an outlet controller unit (555) which controls the size of the soldering agent spreader's outlet and/or the flow rate of the soldering agent spreader and/or the spreader-tissue distance.

In a preferred embodiment of the fourth configuration, an agent outlet controller unit (555) transfers signals to a diameter-control motor (565) capable of changing the diameter of the soldering agent spreader's outlet. In another preferred embodiment of the fourth configuration, agent outlet controller unit (555) transfers signals to a regulating valve (580), which is used for regulating the flow rate of the soldering agent.

In yet another preferred embodiment of the fourth configuration, agent outlet controller unit (555) transfers signals to an axial shifting motor (590), which controls the movement of the spreader (510) closer to or farther from the tissue, so as to provide different spreader-tissue distances. The signals mentioned from above are transferred from the computing unit (550) which constantly receives input from the apparatus for determining the spreader-tissue (570), calculates required agent flux changes and implements these changes by controlling one or more of the controlling means mentioned above. For example, when the pace of the soldering system (50) is pre-selected, it is optional to regulate the soldering agent flux by controlling the pace-motor (560), which is responsible for the pace of the system (50). Another example for regulating the agent flux is by controlling the soldering agent flow rate, by sending appropriate signals to the regulating valve (580). Yet another example for regulating the agent flux is by controlling the axial shifting motor (590) which changes the spreader-tissue distance.

The distance between the energy source outlet (523) and the tissue surface, also referred to hereinafter as the laser-tissue distance, is determined by the apparatus (570) for determining the laser-tissue distance. The computing unit (550) constantly receives real-time input of the laser-tissue distance, from which it calculates the energy flux projected on the tissue, and calculates the required changes in energy flux needed for obtaining a desired energy flux. The required energy flux's changes are automatically transferred as signals to appropriate controllers for controlling the size of the soldering beam spot and/or the soldering energy power. The size of the soldering beam spot can be regulated by changing the distance between the optical unit coupled to the energy source and the surface of the tissue. This can be accomplished either by changing the optical focusing of the optical unit or by moving the entire hand piece (520) closer to or farther from the tissue, so as to provide different laser-tissue distances, which includes the energy outlet. The laser-tissue distance is changed by an axial shifting motor (595), which receives signals from the computing unit (550) and moves the laser outlet farther or closer from the tissue.

As mentioned above, in a preferred embodiment of the fourth configuration, the spreader tissue distance and the laser-tissue distance are the same, such that the spreader outlet and the laser outlet are parallel and both are projecting onto the tissue from a hand piece, in such event a hand piece axial-shifting motor which moves the hand piece farther or closer from the tissue, may replace the spreader axial shifting motor (590) and the laser axial shifting motor (595). In yet another preferred embodiment of the fourth configuration, the spreader axial shifting motor (590), the laser axial shifting motor (595) or the alternative hand piece axial-shifting motor are capable of moving the controlled elements coupled and controlled by each motor, (spreader or spreader outlet, laser outlet, hand piece), closer and farther from the tissue in a vertical direction, in a circular horizontal motion and the combination of both.

The pace of the system, the spreader-tissue distance and the laser-tissue distance may be constant or variable, depending on the depth and the width of the wound.

In neurosurgery, it is common practice is to suture peripheral nerves after trauma to reconnect the neural structures and allow sprouting of proximal axons toward the damaged limb through the existing neural canals. The aforementioned soldering system configuration employing a high precision computerized unit (550) may be used for laser welding or soldering of nerves, bundle by bundle. This method and system will enable fast and efficient post-trauma reconstruction of peripheral neural structures.

Reference is made to FIG. 5, illustrating a fifth configuration of a soldering system (60), similar to the fourth configuration, having an additional wound dressing apparatus (690). The wound dressing apparatus (690) adds strength to the closed wound until the natural healing of the tissue is strong enough. The wound dressing is preferably used for an external tissue; accordingly, it is applied on the surface of the wound, to provide another covering and protecting layer. The additional wound dressing helps in preventing the wound from opening during the healing process

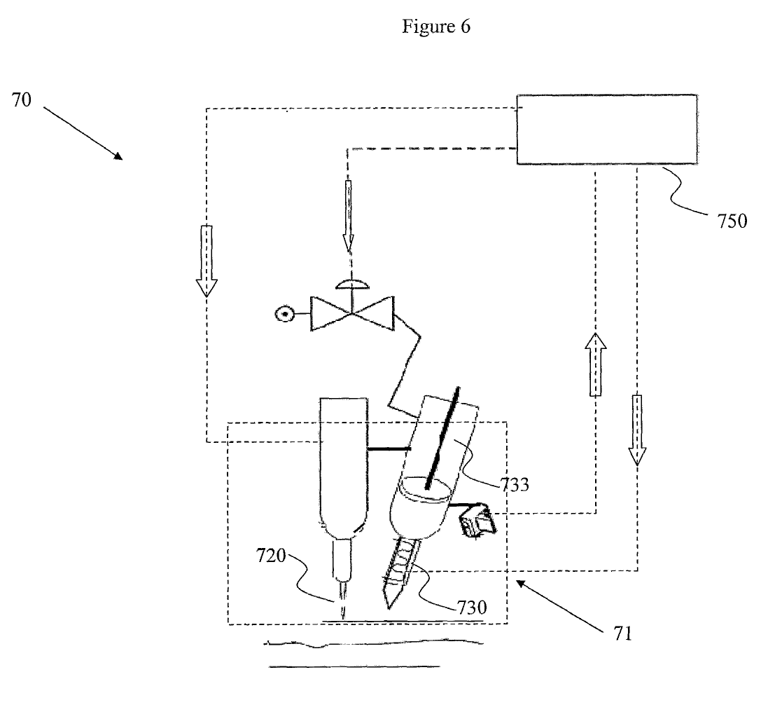

Reference is made to FIG. 6, illustrating a sixth configuration of a soldering system, employing a temperature-controlled soldering agent hand piece. The temperature-controlled soldering hand piece (71) heats the soldering agent flow to a desired temperature as it exits the hand piece towards the tissue surface. The heating of the soldering agent may be accomplished by a heating coil (730) at the tip of the spreader (733). The temperature-controlled soldering hand piece (71) heats the heating coil (730) to a required temperature so as to activate the soldering agent. The temperature of the heating coil is controlled by the computing unit. The temperature of activating the soldering agent, such as albumin, is approximately between 50 degrees Celsius to 70 degrees Celsius.

Reheating and additional activation of the soldering substance occurs when energy power (720) is applied to the tissue surface for soldering.

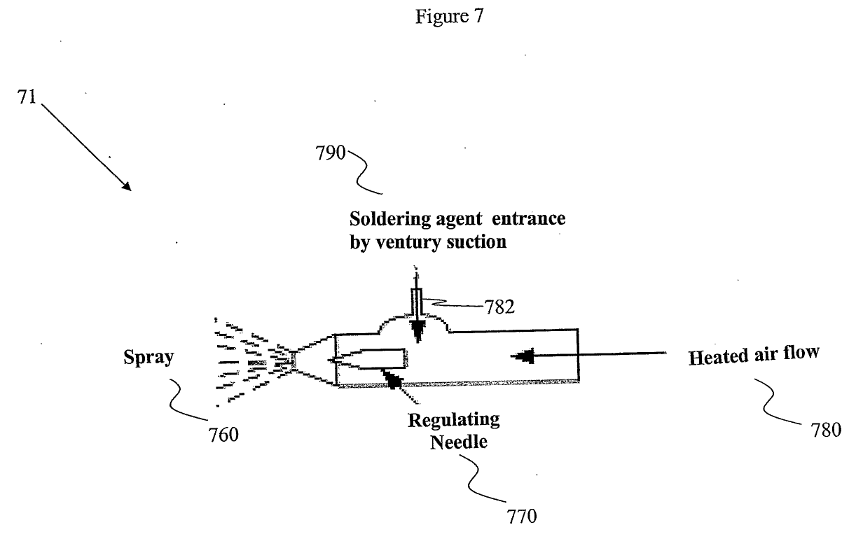

Reference is made to FIG. 7, illustrating an alternative configuration for a temperature-controlled soldering apparatus (71), which heats the soldering agent flow to a required temperature while it is exiting the hand piece towards the surface of a tissue. As the soldering agent (790) enters the temperature-controlled soldering apparatus, as by venturi (782), it is heated by a flow of hot air (780) and directed towards a regulating needle (770) for regulating the heated soldering agent flow exiting the sprayer (760). The temperature-controlled soldering apparatus (71) heats a predetermined amount of air in a tank to a required temperature so as to activate the soldering agent. The temperature of activating the soldering agent, such as albumin, is approximately between 50 degrees Celsius to 70 degrees Celsius.

1-31. (canceled)

32. A computerized system for tissue bonding comprising:
a tissue bonding enhancement agent dispenser operative to dispense tissue bonding enhancement agent onto tissue surfaces to be bonded;
a radiant energy source operative to direct radiant energy onto said tissue surfaces and bonding enhancement agent following dispensing thereof;
a displacer operative to selectably urge said tissue surfaces into close propinquity; and
a computerized controller operative to coordinate operation of at least two of said agent dispenser, said radiant energy source and said displacer.

33. A computerized system for tissue bonding according to claim 32 and wherein said computerized controller coordinates operation of said agent dispenser, said radiant energy source and said displacer.

34. A computerized system for tissue bonding according to claim 32 and also comprising an optical unit operative to gauge a distance of at least one of said radiant energy source and said agent dispenser from said tissue surfaces and to communicate said distance to said computerized controller.

35. A computerized system for tissue bonding according to claim 34 and wherein said optical unit is at least one of a distance sensor and an imaging sensor operative to gauge a spot size projected by a radiant energy beam.

36. A computerized system for tissue bonding according to claim 32 and wherein said computerized controller governs at least one of:
distance of said agent dispenser from said tissue surfaces;
distance of said radiant energy source from said tissue surfaces;
radiant energy flux generated by said energy source;
output flow rate of said bonding enhancement agent from said agent dispenser; and
output temperature of said bonding enhancement agent.

37. A computerized system for tissue bonding according to claim 36 and wherein said computerized controller governs said radiant energy flux by regulating at least one of: beam spot size, beam shape and frequency of said radiant energy source.

38. A computerized system for tissue bonding according to claim 32 and wherein said displacer also assists in maintaining distance of at least one of radiant energy source and said agent dispenser from said tissue surfaces.

39. A computerized system for tissue bonding according to claim 32 and wherein said displacer comprises at least one of a pair of sliders and a pair of wheels.

40. A computerized system for tissue bonding according to claim 39 and wherein said wheels are coupled to a motor operative to rotate said wheels and bring about displacement of at least one of said agent dispenser and said radiant energy source along said tissue surfaces at a predetermined rate of displacement.

41. A computerized system for tissue bonding according to claim 40 and wherein said rate of displacement is gauged by at least one of a tachometer and said motor and communicated to said computerized controller.

42. A computerized system for tissue bonding according to claim 41 and wherein said computerized controller governs said rate of displacement of at least one of said agent dispenser and said radiant energy source along said tissue surfaces.

43. A computerized system for tissue bonding according to claim 42 and wherein said computerized controller coordinates at least two of:
rate of displacement;
dispenser; and
distance of said agent dispenser from said tissue surfaces;
distance of said radiant energy source from said tissue surfaces;
radiant energy flux;
output flow rate of said bonding enhancement agent from said agent output temperature of said bonding enhancement agent.

44. A computerized system for tissue bonding according to claim 32 and also comprising at least one heating coil at a tip of said tissue bonding enhancement agent dispenser.

45. A computerized system for tissue bonding according to claim 32 and also comprising a hot airflow chamber operative to activate said bonding enhancement agent flowing therethrough.

46. A computerized system for tissue bonding according to claim 32 and also comprising a wound dressing dispenser operative to place a wound dressing over said tissue surfaces following bonding thereof.

47. A computerized system for tissue bonding according to claim 32 and wherein said computerized controller and said displacer together provide:
first tissue displacer functionality operative initially to cause displacement of edges of tissue into a mutual non-touching relationship along a seam prior to bonding thereof; and
second tissue displacer functionality operative following operation of said first tissue displacer functionality to cause displacement of said edges into a mutual touching relationship wherein said edges are in mutual propinquity.

48. A computerized system for tissue bonding according to claim 32 and wherein said displacer includes tissue contact elements which are operative for aligning said tissue surfaces to be bonded.
